(19) United States
(12) Patent Application Publication
(10) Pub. No.: US 2001/0029349 A1
(43) Pub. Date: Oct. 11, 2001

(54) Method and apparatus for treating aneurysms

(76) Inventor: Boris Leschinsky, Waldwick, NJ (US)

Correspondence Address: Datascope Corp., 14 Philips Parkway, Montvale, NJ, 07645, US

(21) Appl. No.: 09/880,241

(22) Filed: Jun. 13, 2001

Related U.S. Application Data
(63) Continuation-in-part of application No. 09/165,333, filed on Oct. 1, 1998.
Application No. 09/165,333 is a continuation of application No. 08/631,337, filed on Apr. 12, 1996, now abandoned.

(51) Int. Cl. A61M 29/00

(52) U.S. Cl. 604/101.03

(57) ABSTRACT

A balloon catheter for isolating and treating an aneurysm in a vessel. The catheter including one or more inflatable balloons for defining an isolated volume within the vessel and for preventing any blood flow from coming into contact with the interior walls of the vessel outside the isolated volume. The catheter further including a lumen for injecting into the isolated volume a crosslinking agent, such as glutaraldehyde, for toughening the aneurysmal vessel wall.

### RELATED APPLICATIONS

This application is a continuation-in-part of application Ser. No. 09/165,333, filed on Oct. 1, 1998, which is a continuation of application Ser. No. 08/631,337, filed on Apr. 4, 1996.

### BACKGROUND OF THE INVENTION

1. Field of the Invention

The invention relates to a method and apparatus for repairing an aneurysm.

2. Description of the Prior Art

An aneurysm, such as an abdominal aortic aneurysm, is a sac caused by an abnormal dilation of the wall of the aorta as it passes through the abdomen. The abdomen, located between the thorax and the pelvis, contains a cavity, known as the abdominal cavity, which is separated by the diaphragm from the thoracic cavity. The abdominal cavity is lined with a serous membrane, the peritoneum. The aorta is the main trunk, or artery, from which the systemic arterial system proceeds. It arises from the left ventricle of the heart, passes upward, bends over and passes down through the thorax and through the abdomen to about the level of the two common iliac arteries.

Abdominal aneurysm usually arises in the infra renal portion of the aorta. When left untreated, an aneurysm will eventually cause rupture of the sac with ensuing fatal hemorrhaging in a very short time. High mortality associated with the rupture of the blood vessel has led to the present state of the art and the transabdominal surgical repair of abdominal aortic aneurysms. Surgery involving the abdominal wall, however, is a major undertaking with associated high risks. There is considerable mortality and morbidity associated with this magnitude of surgical intervention, which in essence involves replacing the diseased and aneurysmal segment of blood vessel with a prosthetic device which typically is a synthetic tube or graft.

To perform the surgical procedure, requires exposure of the aorta through an abdominal incision, which can extend from the rib cage to the pubis. The aorta must be clamped both above and below the aneurysm, so that the aneurysm can then be opened and the thrombus, or blood clot, and arteriosclerotic debris removed. Small arterial branches from the back wall of the aorta must also be tied off. The tube or graft, of approximately the same size of the normal aorta, is sutured in place, thereby replacing the aneurysm. The clamps are removed and blood flow is reestablished through the graft.

If the surgery is performed prior to rupturing of the abdominal aorta aneurysm, the survival rate of treated patients is markedly higher than if the surgery is performed after the aneurysm ruptures, although the mortality rate is still quite high.

Disadvantages associated with the conventional, prior art surgery, in additional to the high mortality rate, are: the extended recovery period associated with such surgery; difficulties in suturing the graft or tube to the aorta; and the unsuitability of the surgery for many patients having abdominal aortic aneurysms. As to the extent of recovery, a patient can expect to spend from 1 to 2 weeks in the hospital after the surgery, a major portion of which is spent in the intensive care unit, and a convalescence period at home from 2 to 3 months, particularly if the patient has other illness such as heart, lung, liver, and/or kidney disease, in which case the hospital stay is also lengthened. Another difficulty involved in performing the suturing step in the presence of a clot on the remaining portion of the aorta, as well as situations where the remaining portion of the aorta often becomes friable, or easily crumbled.

Since the clot is typically removed in the prior art surgery, the new graft may not have the benefit of the previously existing thrombosis therein, which may actually reinforce the walls of the vessel if the graft was able to be inserted within the existing clot. Since many patients having abdominal aortic aneurysms are older and have other chronic illnesses, such as heart, lung, liver, and/or kidney disease, they are not ideal candidates for such major surgery. Such patients have difficulties in surviving the operation.

It has been previously proposed to repair abdominal aortic aneurysms by intraluminal delivery of an aortic graft disposed upon a catheter, and securing the graft within the aorta by expansion and deformation of an expandable deformable member associated with the graft by expanding and inflating a portion of the catheter which contacts the tubular member. Because of the relatively large diameter of the catheter and associated graft necessary for implantation within the aorta, some difficulties have been encountered. Problems encountered include spasms associated with the access body vessel such as the femoral artery and kinking of the graft during or after implantation. There are also problems associated with stent/grafts including leaks which spring between the vessel wall and the graft.

An alternate repair method is transluminal deployment of the bifurcated stent/graft. It has been under development by many investigators for the last 10 years. A large variety of designs are being evaluated at the present time. The method for implantation of the bifurcated stent/graft is also known in the art. In spite of some differences between approaches, all of them have the same basic principle: the vascular graft is deployed through the femoral artery to isolate the sac of the aneurysm and restore the natural shape and patency of the vessel tree.

The graft is reinforced by a metal (typically, stainless steel or a super elastic metal) stent. The stent aids in attachment of the graft to the vessel wall and also prevents kinking. The device can be made as one piece or can consist of two or three parts that are connected to each other inside the patient.

Advantages of transluminal deployment are the avoidance of highly invasive surgery and the reduction of bleeding risks. Mains concerns, however, include: (a) difficulties and complications encountered in insertion manipulation; (b) the existence of a great variety of aneurysmal sac and healthy vessel geometries; and (c) difficulties encountered in attaching and sealing the graft to that arterial wall.

### SUMMARY OF THE INVENTION

It is an object of this invention to provide a method and apparatus for the percutaneous treatment of aneurysms.

Another object of this invention is to provide a method and apparatus for treating aneurysms located at a vessel bifurcation.

A still further object of the invention is to prevent rupture of the arterial wall by changing the nature and structure of the vessel wall.

In accordance with one aspect of this invention, an aneurysm in a vessel is treated by first isolating, with at least one percutaneously administered expandable balloon, a volume in the vessel around the aneurysm. Any biological debris trapped within the isolated volume may then be removed by infusion and aspiration with a flushing fluid. A cross linking substance is then placed into the isolated volume to aide in the strengthening and toughening of the vessel wall. Once the wall is crosslinked, and thus toughened, the balloons are deflated and removed to allow normal flow of blood through the vessel.

U.S. Pat. Nos. 5,213,580, 5,328,471, 5,575,815, 5,500,538, 5,662,609, 5,634,946, 5,674,287, 5,749,915, 5,749,922, 5,947,977, and WO96/11021 issued to Slepian et al., disclose a catheter system for paving or coating the inner surface of a blood vessel. The biodegradable coating allows the blood vessel to heal after an angioplasty procedure and also helps prevent restenosis. A disadvantage of the coating is that it is biodegradable, and thus, cannot serve a vessel wall strengthening function, if at all, for extended periods of time.

The various objects, advantages and novel features of this invention will be more fully apparent from a reading of the following detailed description in conjunction with the accompanying drawings in which like reference numerals refer to like parts.

### BRIEF DESCRIPTION OF THE DRAWINGS

FIG. 1 is a longitudinal cross section of an aneurysmal artery and surround tissue.

FIG. 2 is a side view of one embodiment of the invention inserted into the aneurysmal artery of FIG. 1.

FIG. 3 is a view, partly in schematic and partly in perspective form of portions of the apparatus taken along lines 3-3 of FIG. 2.

FIG. 4 is a longitudinal cross sectional view of a typical abdominal aortic aneurysm with the balloon catheterization in place and a closed flushing system contained within the catheterization system in accordance with one embodiment of the invention.

FIG. 4A is a transverse cross sectional view of the leg of the Y-shaped catheter.

FIG. 4B is a transverse cross sectional view of the left arm of the Y-shaped catheter.

FIG. 4C is a transverse cross sectional view of the right arm of the Y-shaped catheter.

FIG. 5 is a longitudinal cross sectional view of a typical abdominal aortic aneurysm with the balloon catheter in place and an open flushing system contained within the catheterization system in accordance with another embodiment of the invention.

FIG. 5A is a transverse cross section of the catheter of FIG. 5 proximal pump 138.

FIG. 5B is a transverse cross section of the catheter of FIG. 5 distal pump 138.

FIG. 6 is a longitudinal cross sectional view of the catheter of FIG. 4 having additional branches for occlusion of the renal arteries.

FIG. 6A is a transverse cross section of the catheter of FIG. 6 proximal pump 138.

FIG. 6B is a transverse cross section of the catheter of FIG. 6 distal pump 138.

FIG. 7 is a longitudinal cross sectional view of the aortic aneurysm excluded by a stent/graft device.

### DESCRIPTION OF ILLUSTRATIVE EMBODIMENTS OF THE INVENTION

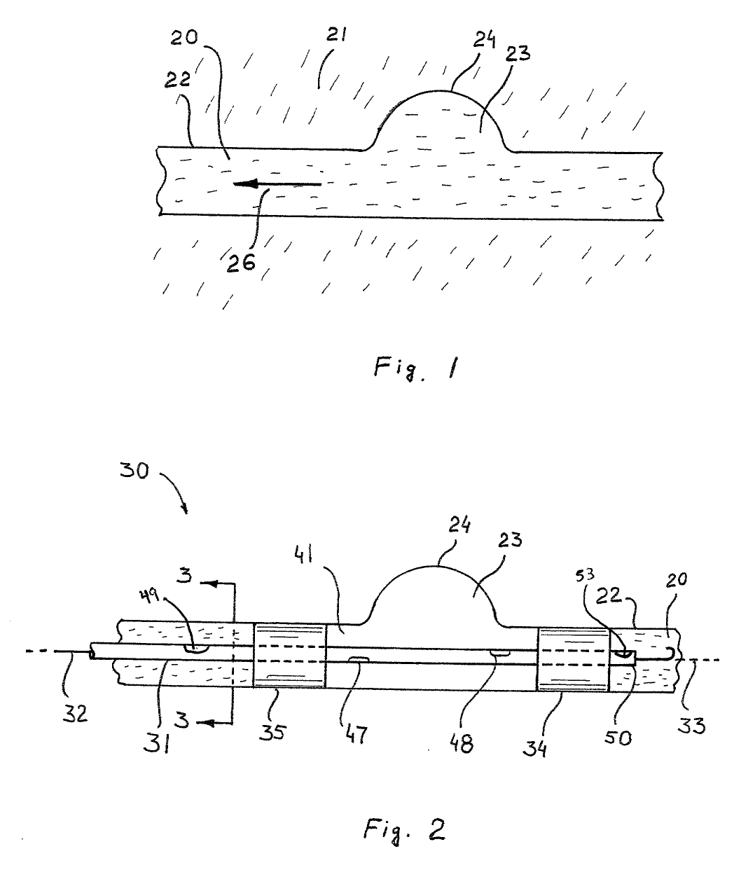

The words “proximal” and “distal” as used below have the following meaning, the proximal end of the catheter device is the end inserted into the patient first via a percutaneous insertion. For example, in FIG. 2, the most proximal portion of the catheter device is tip 50. The invention will now be described with respect to the figures. FIG. 1, in simplified form, illustrates a single-passage, tubular vessel 20 through tissue 21, such as peri-arterial tissue, defined by a vessel wall 22. Although FIG. 1, and the other figures, depict a vessel wall as comprising a single homogeneous layer, it will be recognized that an actual vessel wall has multiple layers. However, this invention can be understood by referring to the simplified, homogenous representation in the figures. In addition, and as later to be discussed, vessel 20 maybe a bifurcated vessel such as the abdominal aortic.

FIG. 1 illustrates an aneurysm 23 in vessel wall 22 that is an abnormal dilation of blood vessel 20 due to weakening and stretching of an aneurysmal wall 24 in otherwise normal wall portion 22. Blood flows in a direction represented by arrow 26 within vessel 20. If left untreated, the aneurysm 23 can grow in size, rupture anal allow hemorrhaging of blood from vessel 20 into the surrounding tissue or cavity 21.

FIG. 2 depicts a side view of system 30, inserted in vessel 20 of FIG. 1, comprising a catheter 31 positioned over a percutaneously administered guidewire 32. Catheter 31 extends generally along an axis 33 and supports a proximal occlusion balloon 34 and an axially spaced distal occlusion balloon 35.

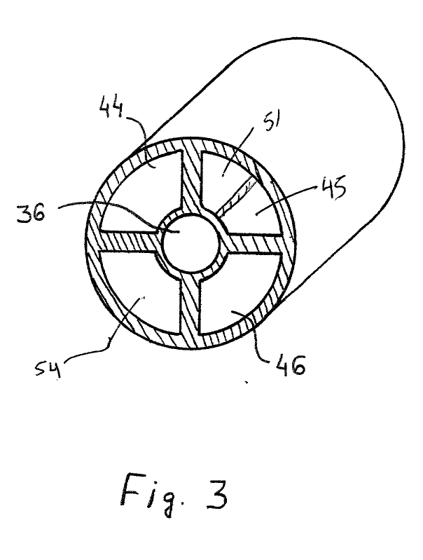

Referring to FIGS. 2 and 3, catheter 31 also includes a central guidewire lumen 36 and occlusion balloon inflation lumens 45 and 51 that connect to a distal occlusion balloon inflation source (not shown). FIG. 2 depicts device 30 after the occlusion balloon inflation source expands balloons 34 and 35 in vessel 20 against healthy portions of the wall 22 proximally and distally of aneurysm 23. Occlusion balloons 34 and 35 thereby define an isolated volume 41 in the vessel 20 around the aneurysm 23.

A remote distal vacuum source (not shown) connects to a suction lumen 46 that terminates at port 47 located distally of the proximal occlusion balloon 34. Alternatively, port 47 can be located at any location intermediate occlusion balloons 34 and 35. When the vacuum source applies suction to lumen 46, it draws blood in vessel 20 through lumen 46, and thereby, evacuates isolated volume 41. At this point in the sequence, the occlusion balloons 34 and 35 are still expanded to define the isolated volume 41.

While the specific apparatus 30 in FIG. 3 includes catheter 31 with multiple discrete lumens, certain functions of these lumens may be combined in a single lumen, for example, the vacuum source might connect directly to the guidewire lumen to evacuate blood in isolated volume 41 through guidewire lumen 36 over guidewire 32. Other such functional combinations are also possible. In addition, each of the individual components including the balloons 34 and 35 and catheter 31 have conventional constructions. Furthermore, choice of particular lumens in catheter 31 for suction, infusion, inflation, and deflation is arbitrary.

Once occlusion balloons 34 and 35 are positioned, infusion of an optional flushing fluid, such as saline, may be made through lumen 44 and out infusion port 48. Loosened particles of friable material and excess fluid are removed from treatment chamber 41 back through lumen 46 for removal from system 30. Next, a crosslinking chemical solution is pumped through lumen 44 and port 48 into the treatment chamber 41. The solution is optionally allowed to sit in the treatment chamber 41 for a predetermined amount of time after which it is pumped out via port 47 and lumen 46. During the above treatment blood flow is maintained. Blood enters port 51, flows through lumen 54, and exits port 53, thus, bypassing aneurysm 24.

The purpose of the chemical solution is to strengthen aneurysmal wall 23 by actually changing the nature of the wall 23, i.e. crosslinking the collagen in the wall 23. While various classes of chemical solutions can be used to strengthen or reinforce the wall 22 of the artery 20, the preferred solutions are aldehydes and especially glutaraldehyde, since aldehydes are proven cross linking agents routinely used for preparation and disinfection of animal tissues (e.g., porcine valves and blood vessels) before implantation in humans. The main effect of crosslinking is to “toughen” weakened vessel wall 22.

Another possible crosslinking agent is carbodiimide which has the advantage of being more biocompatible and does not have the toxicity of a glutaraldehyde. Other classes of chemical agents may be considered. They may even be toxic since no such fluid is allowed to migrate from the isolated treatment chamber 41. Because the blood continues to flow through lumen 54, there is no time constraints placed on the flushing of the treatment chamber 41.

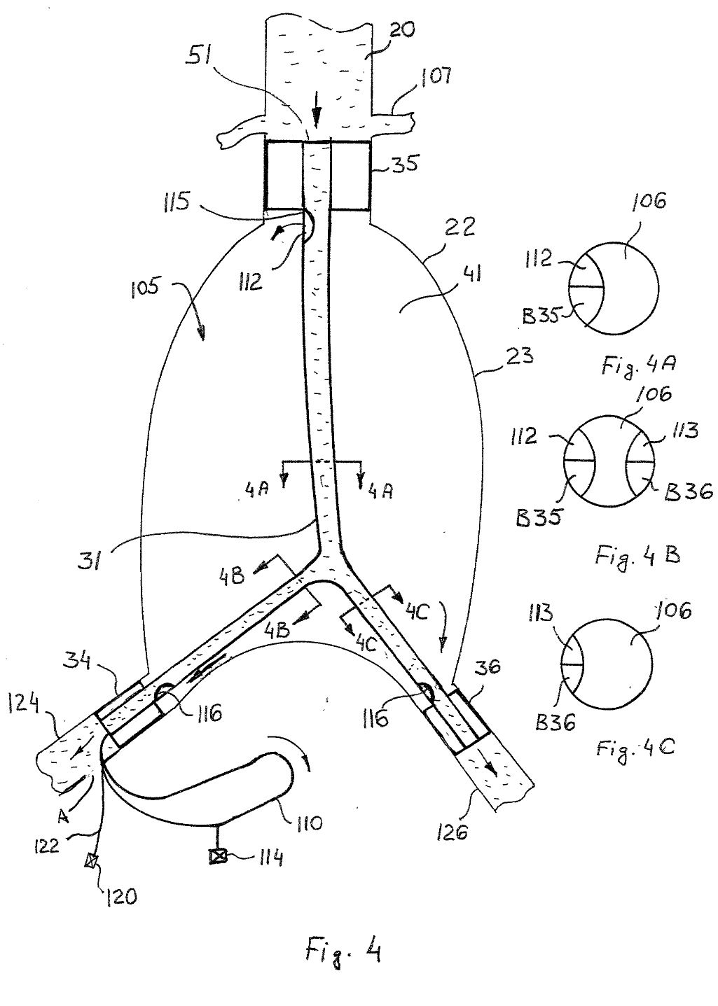

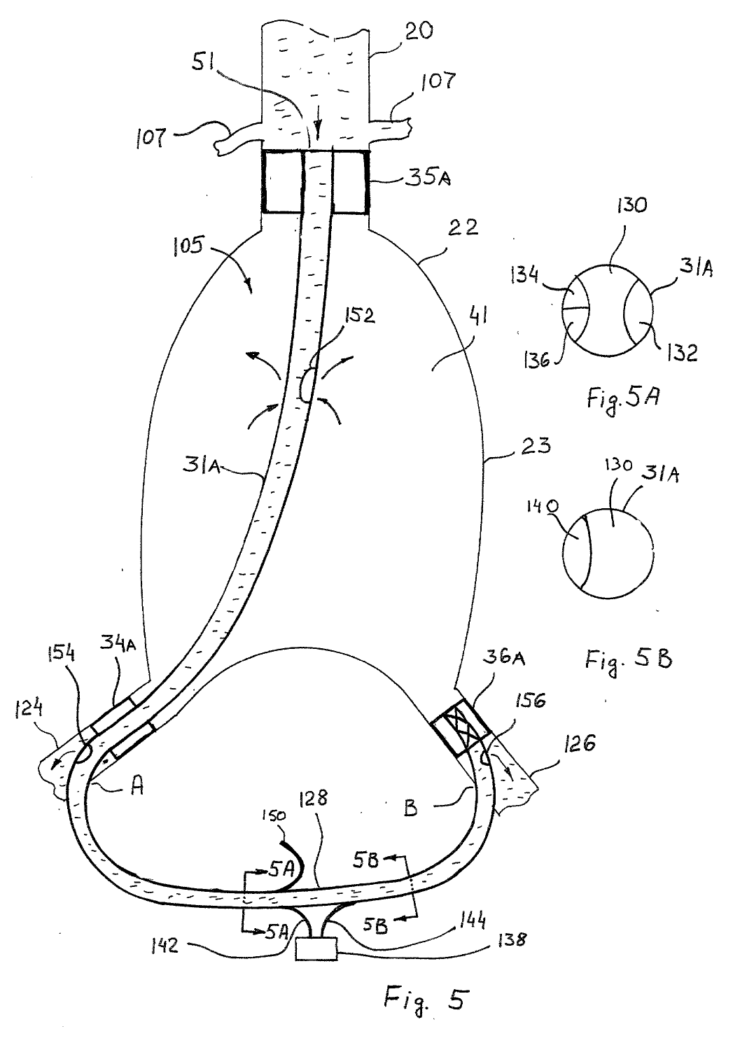

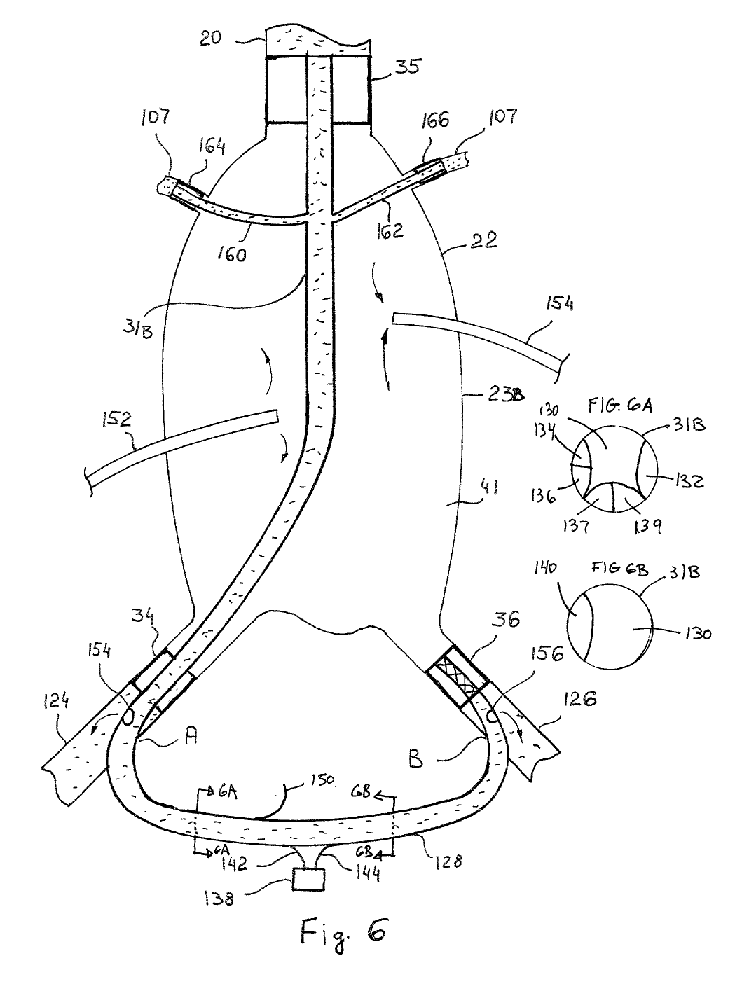

FIGS. 4 and 5 illustrate another embodiment of the invention which can be used to treat an abdominal aortic aneurysm (“AAA”). A preliminary step may involve closure of secondary vessels adjacent the aneurysm. Commonly known techniques, to prevent chemical solution used in the procedure from traveling to other areas of the body, may be employed. Furthermore, commonly known techniques, similar to those used to insert bifurcated grafts, may be used to percutaneously insert the catheters illustrated in FIGS. 4, 5, and 6.

FIG. 4 illustrates an isolation device 105 consisting of a series of occluding balloons 34, 35 and 36, connected to Y-shaped catheter 31, which upon insertion and inflation together with an inner surface of the diseased vessel wall 22 define a treatment chamber 41 within an aneurysm 23 in the abdominal aorta 20. Catheter 31 is inserted through insertion site labeled A. Insertion of balloons 34, 35 and 36 is performed such that the proximal occluding balloon 35 is positioned first in the abdominal aorta 20 and inflated just below the renal arteries 107 in the healthy section of abdominal aorta 20, proximal diseased vessel wall 22. Following this step, two iliac or femoral occluding balloons 34 and 36 are positioned and inflated in corresponding arteries just below the end of treatment chamber 41. Catheter 31 defines a lumen 106 (FIG. 4A) which allows blood to bypass aneurysm 23 and flow to the legs of a patient during the procedure. Note that catheter 31 is shown filled with blood. Occluding balloons 34, 35 and 36 are made with conventional procedures and materials and are soft enough to allow for good hydraulic isolation of treatment chamber 41 while being sufficiently strong to prevent migration downstream under pressure. Fluid or gas used in inflation of balloons 34, 35 and 36 maybe any of the conventional gases or fluids used in inflating balloon within the body of a patient, such as saline or an inert gas.

Upon achieving isolation of the treatment chamber 41, chamber 41 is flushed with an appropriate solution. Solution fluid is introduced via a fluid circuit consisting of a fluid reservoir 114, external lumen 111 (not shown), defined by external solution tube 110, flush lumen 112 in catheter 31, see FIGS. 4A and 4B, and vacuum lumen 113 in catheter 21, see FIGS. 4B and 4C. Solution, examples of which were discussed earlier, is circulated by a pump (not shown), or other means known in the art for circulating fluids, from the fluid reservoir 114, through external lumen 111 and flush lumen 112, out flush port 112 into treatment chamber 41, out vacuum ports 116 through vacuum lumen 113 and back to external lumen 111 for reintroduction into treatment chamber 41. Note that flush rate and duration of the flush will vary depending on the size of aneurysm 23 and the desired level of coating or crosslinking. Note that ports 112 and 116 may be located anywhere in treatment chamber 41 along catheter 31 and that use of a different number of ports is anticipated. Furthermore, the location and arrangement of lumens located within, connected to, or embedded in catheter 31 is not critical to this invention. Various lumen arrangements can be use and a single lumen can be used for multiple tasks.

Balloon 34, 35, and 36 are inflated via a pump circuit comprising a pump 120 connected to catheter 31 by means of an external tube 122. External tube 122 defines an external lumen 119 (not shown) which communicates with lumens B35 and B36, see FIGS. 4 and 4A-4C, for inflation and deflation of balloons 34, 35, and 36.

FIG. 5 illustrates another alternative embodiment of the invention comprising catheter 31A and occlusion balloons 34A, 35A, and 36A. One benefit of this embodiment is the ease of insertion compared to the embodiment illustrated in FIG. 4 which requires manipulation of the catheter from the right common iliac 124 to the left common iliac 126. As illustrated in FIG. 5, the proximal end of catheter 31 is advanced into the aorta 20 through an insertion site labeled A and just past aneurysm 23. Balloon 35A is inflated such that the proximal end of catheter 31 is fixed just distal or below renal arteries 107. Balloon 34A is inflated and fixed in the right common iliac 124 just proximal or above insertion site A. A distal end of catheter 31 is then advanced through insertion site labeled B into the left common iliac 126. Balloon 36A is then inflated and fixed in the left common iliac 126. Portion 128 of catheter 31 remains outside of the patient's body.

As illustrated in FIGS. 5A and 5B, catheter 31 has a blood bypass lumen 130, an infusion/vacuum lumen 132, an inflation/deflation lumen 134 for balloon 35A, an inflation/deflation lumen 136 for balloon 34A, and an inflation/deflation lumen 140 for balloon 36A. A pump 138 for inflating and deflating balloons 34A, 35A, and 36A is connected to inflation/deflation lumen 136 and inflation/deflation lumen 134 by tube 142 and is connected to inflation/deflation lumen 140 by tube 144. Note that pump 138 may be replaced with any device known in the art capable of inflating and deflating balloons 34A, 35A, and 36A, including a syringe.

Upon placement of catheter 31 and inflation of balloons 34A, 35A, and 36A treatment chamber 41 is optionally flushed with a flushing solution, such as saline. The flushing solution is pumped through tube 150 by a pump (not shown) or other means known in the art through communicating infusion/vacuum lumen 132 and port 152 into treatment chamber 41. The flushing solution is then removed from the treatment chamber via the same port 152. Alternatively, different ports and lumens can be used for infusion and removal of solution. Next, a chemical solution, preferably glutaraldehyde, other examples of which were described and listed in reference to first and second embodiments, is pumped through tube 150, infusion/vacuum lumen 132 and port 152 into treatment chamber 41. As indicated above the chemical solution actually changes the nature of wall 22. Next, the chemical solution is pumped out of port 152, through infusion/vacuum lumen 132, and out tube 150. The flushing and chemical solution infusion cycles may be repeated as necessary. Note that while the therapy is proceeding blood flow to the patient's legs is maintained through lumen 130 in catheter 31. Blood enters the proximal end of catheter 31, by renal arteries 107, and exits through ports 154 and 156. Following treatment with the chemical solution another flushing solution may be employed to remove excess chemical solution from treatment chamber 41.

In yet another alternative embodiment of the invention, illustrated in FIG. 6, the infusion of the flushing solution and the chemical solution into treatment chamber 41 and the removal of said solutions may be done through separate catheters 152 and 154, laparoscopically inserted through aneurysmal wall 22. Unlike aneurysm 23 in FIGS. 4 and 4, aneurysm 23B in FIG. 6 has expanded proximal the renal arteries 107. To prevent the chemical solution from escaping through these arteries catheter 31B is equipped with two arms 160 and 162 having balloons 164 and 166 on their ends which are inflated in, and thereby occlude, each renal artery 107. Catheter 31B is identical to the one illustrated in FIGS. 5, 5A, and 5B except for two additional lumens 137 and 139 used for inflation and deflation of balloons 164 and 166. Arms 160 and 162 may be positioned in the renal arteries 107 using steerable guide wires or any other means known in the art.

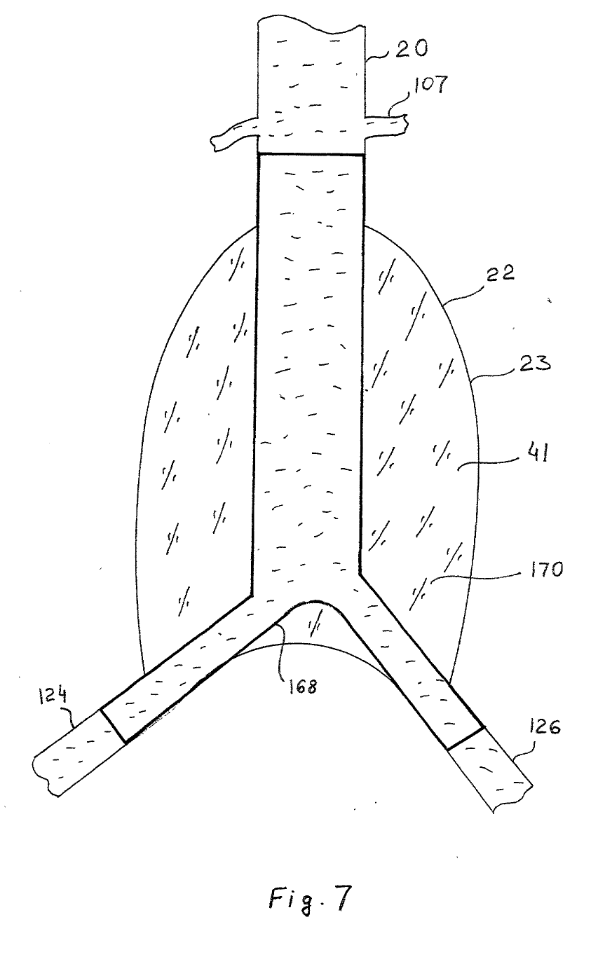

As an alternate method for treating aneurysm 23 or 23B, a stent or stent/graft device 168 can be inserted and deployed in the aneurysm, as illustrated in FIG. 7, and a filling material 170 can then be inserted between the aneurysm wall 22 and the stent or stent/graft device 168. Alternatively, an isolation device having the form of the stent/graft device can be temporarily inserted into the aneurysm and then removed after the filling material solidifies or dries.

In an alternative embodiment of the invention the exterior of the aneurysmal wall of the blood vessel is exposed to the chemical solution. This can be accomplished via a laparoscopic procedure in which a small amount of the chemical solution is sprayed onto or otherwise applied to the aneurysmal wall and optionally adjacent portions of the blood vessel.

It is also anticipated to utilize the chemical solution of the present invention to strengthen or toughen intracranial or brain aneurysms. Various methods and devices exist for treating intracranial aneurysm, see for example U.S. Pat. No. 5,895,385, which involves leaving a small wire or coil in the aneurysm in order to induce thrombus formation in the aneurysm thereby preventing rupture. This and similar methods, share a common disadvantage: they require the aneurysmal blood vessel to be completely blocked off. The present invention overcomes this inherent disadvantage of the prior art by strengthening or toughening the aneurysmal blood vessel as opposed to completely blocking it off. A small amount of the chemical solution, varying depending on the size of the aneurysm but roughly one quarter (¼) to two (2) cubic centimeters, may be injected directly around the blood vessel. A hypodermic needle or other means known in the art for accessing the outer surface of intracranial blood vessels may be used to deliver the chemical solution, which may comprise any of the above listed solutions in relation to the first and second embodiments of the invention. Alternatively, a miniaturized version of catheter 31 or 31A illustrated in FIGS. 2 or 4, respectively, may be used.

From the above it is apparent that many modifications can be made to the disclosed apparatus and method without departing from the invention, such as using mechanical means other than balloons that expand once in position and contract after treatment of the aneurysm is completed or using a microcatheter to access intracranial blood vessels. Therefore, it is the intent of the appended claims to cover all such variations and modifications as come within the true spirit and scope of this invention.

I claim:

1. A device for treating an aneurysm in the wall of a bodily vessel comprising an elongated body having a longitudinal axis and defining at least one lumen along said longitudinal axis, a distal end of said elongated body being connected to a source of crosslinking solution and a means for pumping said crosslinking solution from said source through said lumen out a port toward the proximal end of the elongated body for crosslinking at least a portion of the vessel.

2. The device as claimed in claim 1 wherein the crosslinking solution is an aldehyde solution.

3. The device as claimed in claim 1 wherein the crosslinking solution is a glutaraldehyde solution.

4. The device as claimed in claim 1 wherein the crosslinking solution is carbodiimide.

5. The device as claimed in claim 1 wherein the elongated body is a catheter.

6. The device as claimed in claim 5 wherein the catheter further comprises an occlusion means for isolating the aneurysm.

7. The device as claimed in claim 6 wherein the occlusion means comprises two or more balloon membranes connected to the catheter and spaced a predetermined distance apart.

8. A balloon catheter for treating an aneurysmal wall of a bodily vessel, said catheter defining one or more lumens for inflation and deflation of two spaced apart balloon membranes connected to the catheter and defining one or more lumens for infusion of a crosslinking solution through one or more ports in the catheter between said balloon membranes for crosslinking the aneurysmal wall, a distal end of the catheter being connected to a crosslinking solution reservoir.

9. The balloon catheter as claimed in claim 8 wherein the crosslinking solution is an aldehyde solution.

10. The balloon catheter as claimed in claim 8 wherein the crosslinking solution is a glutaraldehyde solution.

11. The balloon catheter as claimed in claim 8 wherein the crosslinking solution is carbodiimide.

12. A method for treating a weakened portion of a vessel having an inner surface comprising the steps of:
(a) isolating the weakened portion of the vessel;
(b) passing an isolation device having an outer surface through the weakened portion of the vessel;
(c) filling the area between the inner surface of the weakened portion of the vessel and the outer surface of the catheter with a filling material; and
(d) removing the isolation device from the weakened portion of the vessel.

13. The method as claimed in claim 12 wherein the isolation device is a stent graft.

14. The method as claimed in claim 12 wherein the filling between the inner surface of the weakened portion of the vessel and the outer surface of the catheter with a filling material forms a blood passage way.

15. A method for treating an aneurysm in the wall of a bodily vessel defined by an aneurysmal wall with adjacent normal wall portions, said method comprising the steps of:
(a) inserting an elongated body into the blood vessel, said elongated body having a longitudinal axis and defining at least one lumen along said longitudinal axis and having at least one port;
(b) advancing said elongated body to a location wherein the port is near the aneurysm; and
(c) injecting crosslinking solution through said lumen out of the port into the blood vessel such that it contacts the aneurysmal wall.

16. The method as claimed in claim 15 wherein crosslinking solution is an aldehyde.

17. The method as claimed in claim 15 wherein the crosslinking solution is glutaraldehyde.

18. The method as claimed in claim 15 wherein the crosslinking solution is carbodiimide.

19. The method as claimed in claim 15 wherein the elongated body is a catheter.

20. The method as claimed in claim 15 wherein the elongated body is a balloon catheter having spaced apart balloon membranes and wherein prior to injecting the crosslinking solution through the port between the balloon membranes, the balloon membranes are inflated on both sides of the aneurysm and contact the vessel wall so as to seal off the aneurysm from the rest of the vessel.

21. A method for treating an aneurysm in the wall of a bodily vessel defined by an aneurysmal wall with adjacent normal wall portions, said method comprising the steps of:
(a) inserting a catheter into the vessel, said catheter defining one or more lumens for inflation and deflation of two spaced apart balloon membranes connected to the catheter and defining one or more infusion/vacuum lumens for infusion or removal of one or more solutions through one or more infusion/vacuum ports in the catheter between said balloon membranes;
(b) positioning the catheter such that the balloon membranes are on opposite sides of the aneurysm;
(c) inflating both balloon membranes such that the balloon membranes and the aneurysmal wall define a treatment chamber which is isolated from the rest of the vessel, the balloon membranes upon inflation contact the vessel wall;
(d) infusing a crosslinking solution through the infusion/vacuum lumen into the treatment chamber; and
(e) removing the crosslinking solution from the treatment chamber.

22. The method as claimed in claim 21 further comprising the step of infusing a flushing solution through the infusion/vacuum port into the treatment chamber and removing said flushing solution from the treatment chamber through said infusion/vacuum port prior to infusing the crosslinking solution.

23. A method for treating an aneurysm in the wall of a bodily vessel defined by an aneurysmal wall with adjacent normal wall portions, said method comprising the steps of:
(a) isolating, with an isolation means, a volume in the vessel around the aneurysm;
(b) injecting a crosslinking solution into the volume;
(c) clearing the isolated volume of the crosslinking solution; and
(d) removing the isolation means.

24. The method as claimed in claim 23 further comprising the steps of injecting a flushing solution into the volume and removing said flushing solution prior to injecting the crosslinking solution.

25. The method as claimed in claim 23 wherein the isolation means comprises a balloon catheter having two spaced apart balloon membranes and wherein the crosslinking solution is an aldehyde solution.

26. A method for treating an aneurysm in the wall of a bodily vessel defined by an aneurysmal wall with adjacent normal wall portions, said method comprising the steps of:
(a) laparoscopically accessing an exterior surface of the aneurysmal wall; and
(b) applying a crosslinking solution to the exterior surface of the aneurysmal wall.

27. The method as claimed in claim 26 wherein crosslinking solution is an aldehyde.

28. The method as claimed in claim 26 wherein the crosslinking solution is glutaraldehyde.

29. The method as claimed in claim 26 wherein the crosslinking solution is carbodiimide.

30. A method for treating a brain aneurysm defined by an aneurysmal wall with adjacent normal wall portions, said method comprising the steps of:
(a) inserting a needle into the brain such that a tip of said needle is adjacent an exterior wall of the brain aneurysm;
(b) injecting a crosslinking solution onto the exterior surface of the aneurysmal wall.

31. The method as claimed in claim 30 wherein crosslinking solution is an aldehyde.

32. The method as claimed in claim 30 wherein the crosslinking solution is glutaraldehyde.

33. The method as claimed in claim 30 wherein the crosslinking solution is carbodiimide.

34. The method as claimed in claim 23 wherein the crosslinking solution is injected into the volume by means of a cannula, said cannula being inserted laparoscopically such that a distal end of said cannula is inside the aneurysm.
